(19) United States
(12) Patent Application Publication
(10) Pub. No.: US 2008/0286826 A1
(43) Pub. Date: Nov. 20, 2008

(54) Luminescent Particle and Method of Detecting a Biological Entity Using a Luminescent Particle

(75) Inventors: Peter Jan Van Der Zaag, Waalre (NL); Erik Petrus Antonius Maria Bakkers, Heeze (NL); Martinus Bernardus Van Der Mark, Best (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/094,611

(22) PCT Filed: Nov. 20, 2006
(86) PCT No.: PCT/IB06/54330

§ 371 Date: May 22, 2008

(30) Foreign Application Priority Data
Nov. 22, 2005 (EP) 05111075.7

(51) Int. Cl. C12Q 1/02; C09K 11/77; G01N 33/566; C09K 11/70

(52) U.S. Cl. 435/29; 252/301.6 R; 252/301.4 P; 436/501

(57) ABSTRACT

The invention provides a luminescent particle (10) and a method of detecting a biological entity using a luminescent particle, the luminescent particle comprising a core area (20) and a shell area (30), the core area (20) being covered by the shell area (30), the core area (20) conferring a luminescent behavior on the luminescent particle (10) for at least one excitation wavelength and for at least one emission wavelength by means of a nanocrystal material (21), and the shell area (30) being provided such that it realizes an antireflective coating (31) of the core area (20).

The present invention relates to a luminescent particle and a method of detecting a biological entity using a luminescent particle.

The present invention discloses a luminescent particle. Organic dyes, such as fluorescent molecules, have been used to label biological materials. These fluorochromes or fluorophores, however, have several disadvantages. For example, fluorochromes generally have narrow wavelength bands of absorption (e.g., about 30-50 nm), broad wavelength bands of emission (e.g., about 100 nm), and broad tails of emission (e.g., another 100 nm) on the red side of the spectrum. Due to the wavelength properties of these fluorophores, the ability to use a plurality of different colored fluorescent molecules is severely impaired. Furthermore, the fluorescence is extremely susceptible to photobleaching. Nanometer-size semiconductor particles (nanoparticles) are particles which exhibit quantum confinement effects in their luminescent properties. These semiconductor nanoparticles are also known as “quantum dots.” Colloidal particles containing quantum dots can be excited by a single excitation source, providing extremely robust, broadly tunable nanoemitters. In addition, the nanoparticles exhibit optical properties which are superior to those of organic dyes. Their distinctive luminescent properties give quantum dots the potential for a dramatic improvement of the use of fluorescent markers in biological studies. Quantum dots are generally known, e.g. from US-Patent application US 2004/0033345 A1.

Recently, the interest in optical imaging for medical application has been rising as optical imaging holds the promise of non-invasive imaging using fairly inexpensive equipment with high resolution. This appears to be a very useful approach especially for the staging of cancer therapy. There too, the advantages of luminescent particles comprising quantum dots can be of very high relevance, e.g. the advantage over known organic dyes that nanoparticle quantum dots do not photobleach, i.e. degrade under illumination, which is very relevant in 3D imaging, where parts will be irradiated over a long time. Furthermore, they have a larger absorption cross-section, which makes quantum dots brighter than dyes. Moreover, they have a long luminescence lifetime, which renders it possible to separate autologous luminescence for the quantum dot emission by time-gated imaging. However, thus far most of the work has concentrated on Cd-based quantum dots such as CdSe. These are toxic materials which are unlikely to be approved for human applications. A further drawback of known luminescent quantum dot particles is that reflection of the excitation radiation and/or of the emission radiation of the quantum dot causes a considerable loss in luminescence properties. This strongly limits the possibility of using quantum dot luminescent particles e.g. as contrast agents in biomedical applications.

It is therefore an object of the present invention to provide a luminescent particle, a contrast agent, a method of detecting a biological entity using a luminescent particle, and a use of a luminescent particle as a contrast agent whereby the luminescence properties of the luminescent particle are enhanced.

The above object is achieved by a luminescent particle comprising a core area and a shell area, the core area being covered by the shell area, the core area conferring a luminescent behavior on the luminescent particle for at least one excitation wavelength and for at least one emission wavelength by means of a nanocrystal material, and the shell area being provided such that it realizes an antireflective coating of the core area.

This has the advantage that a higher percentage of the excitation radiation energy is potentially available as emission radiation. This greatly improves the emission and reduces reflection losses.

According to the present invention, it is very much preferred that the core area is provided as a quantum dot structure realized by means of a non-Cd-based nanocrystal material and/or that the core area is provided as a quantum dot structure realized by means of a non-toxic nanocrystal material. This renders it possible to provide a material as quantum dot material which can be used in vivo, e.g. as a contrast agent.

Furthermore, it is preferred according to the present invention that the core area is provided as a quantum dot structure realized by means of an InP-based-material. InP as the material for the core area would provide a highly performing quantum dot luminescent particle. It is possible to judiciously choose the size of the core area, i.e. the size of the quantum dot, e.g. around 7 nm in diameter, and thereby tune the excitation and the emission frequency or the excitation and the emission wavelength of the luminescent particle.

Very preferably according to the present invention, the excitation wavelength and the emission wavelength are provided in the near infrared portion of the electromagnetic spectrum. It is possible then to use cost-effective radiation sources for providing the excitation light and also cost-effective radiation detectors e.g. for medical applications in vivo.

It is further preferred according to the present invention that the excitation wavelength and the emission wavelength are provided in a spectral window of minimum infrared absorption in main components of human tissue, especially in human tissue liquid and/or lipid. Very preferably, the excitation wavelength and the emission wavelength are provided between 700 nm and 800 nm. This renders it possible to use the luminescent particle as contrast agents or at least as part of a contrast agent, e.g. for medical use.

It is preferred according to the present invention that the excitation wavelength is provided around 720 nm and/or the emission wavelength is provided around 780 nm. This makes it possible to use even more strongly pronounced absorption minima inside the spectral window of minimum infrared absorption in main components of human tissue.

Very preferably according to the present invention, the excitation wavelength and the emission wavelength are separated by approximately 60 nm. This makes it possible to separate easily the emission from the excitation through a simple low-pass filter. The greater this Stokes shift, the easier it will be to separate the excitation from the emission radiation. Furthermore, as there is no self-absorption because the excitation and emission wavelengths are very well separated, the reconstruction of the optical image is simplified as only emission and scattering have to be considered and not absorption and re-emission. Moreover, the absence of self-absorption (in contrast to conventional dyes) makes it possible to operate at higher concentrations than is possible with dyes. These higher concentrations in their turn lead to an improvement of the emission radiation quality. Improved emission is especially important for the use of the luminescent particle as a contrast agent or as part of a contrast agent, because this renders it possible to image objects situated deeper into the body.

According to the present invention, it is very much preferred that the thickness of the shell area is provided approximately homogeneous around the core area. This provides a better excitation and emission behavior because the reflection of the excitation and/or emission radiation is better controllable.

Furthermore, it is preferred according to the present invention that the thickness of the shell area is provided such that the reflectance of radiation of the excitation wavelength and/or the reflectance of radiation of the emission wavelength between the inside of the core area and the outside of the luminescent particle is comparably low. The unfavorable effects of reflections of the radiation entering and/or leaving the core area of the luminescent particle can be minimized thereby, so that the quality of the emission radiation is enhanced.

Very preferably according to the present invention, the thickness of the shell area is at least partially provided such that the reflectance of radiation of the excitation wavelength and/or the reflectance of radiation of the emission wavelength between the inside of the core area and the outside of the luminescent particle is comparably low. The unfavourable effects of reflections of the radiation entering and/or leaving the core area of the luminescent particle can be at least partially minimized thereby, so that the overall quality of the emission radiation is enhanced.

It is further preferred according to the present invention that the thickness of the shell area is provided such that the transmittance of radiation of the excitation wavelength and/or the transmittance of radiation of the emission wavelength between the inside of the core area and the outside of the luminescent particle is higher than 50% below, preferably higher than 25% below, most preferably higher than 10% below the maximum transmittance for a given first index of refraction of the nanocrystal material, a given second index of refraction of the antireflective coating, and a given third index of refraction of the environment of the luminescent particle.

According to the present invention, it is very much preferred that the shell area comprises a dielectric material and/or that the dielectric material is provided as TiO₂ and/or GaP and/or InGaP₂ and/or other ternary compound(s). This makes it possible to provide the luminescent particle such that a good performance in terms of luminescent behavior of the core area can be combined with excellent properties of the shell area, including a good reflection performance, low toxicity, water solubility, and the possibility to bind a labeling substrate easily to the luminescent particle.

The present invention relates to a complex comprising a luminescent particle according to the embodiments described above and further comprising a labeling substrate. The present invention also relates to a contrast agent comprising an inventive luminescent particle as described above or comprising an inventive complex comprising an inventive luminescent particle and a labeling substrate or comprising a mixture of the present luminescent particle and a complex. Furthermore, the present invention relates to a use of an inventive luminescent particle as described above as a contrast agent or as part of a contrast agent. Such a contrast agent has the advantage that the emission radiation can have a high signal-to-noise ratio, presenting the possibility of a higher optical resolution in imaging techniques.

The present invention also relates to a method of detecting a biological entity using a luminescent particle comprising a core area and a shell area, the core area being covered by the shell area, the core area conferring a luminescent behavior on the luminescent particle for at least one excitation wavelength and for at least one emission wavelength by means of a nanocrystal material, and the shell area being provided such that it realizes an antireflective coating of the core area, the method comprising the steps of:

forming a complex between the luminescent particle and a labeling substrate by means of a physical and/or a chemical and/or a biological binding,

using the labeling substrate for a specific binding to the biological entity,

irradiating at least the complex of the luminescent particle and the labeling substrate with radiation of the excitation wavelength, and

detecting the biological entity by means of radiation emitted by the luminescent particle. This renders it possible to conduct a multitude of different biological assays in an improved manner by means of the luminescent particles according to the present invention.

The present invention also relates to a use of the luminescent particle according to the above embodiments in a biomedical assay and/or an in vitro application. It is to be understood that the inventive luminescent particle can potentially be used together with any biomedical assay format or any in vitro application.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

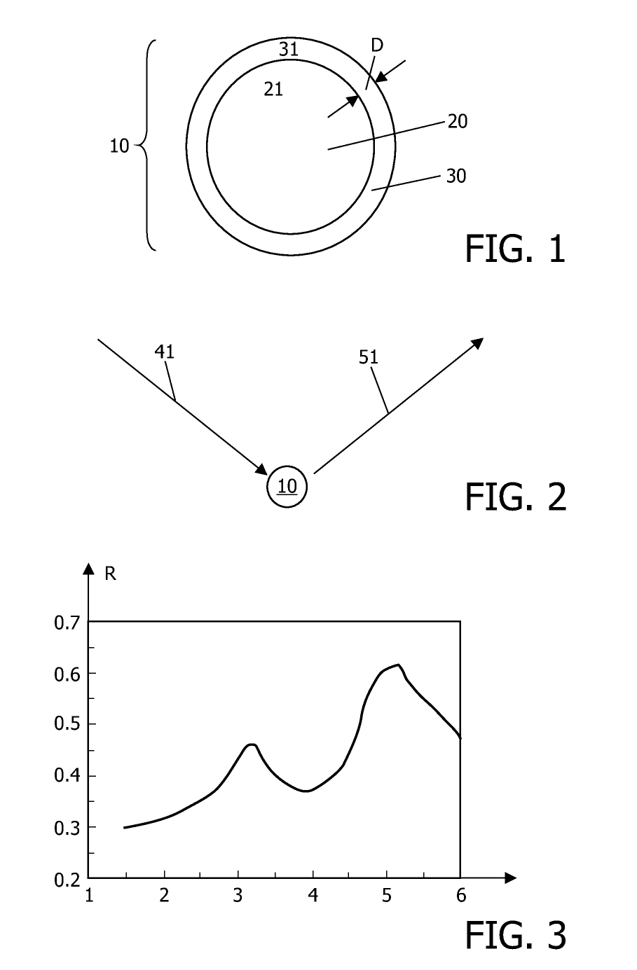

FIG. 1 illustrates schematically a cross-section of a luminescent particle according to the present invention.

FIG. 2 illustrates schematically the luminescent particle according to the present invention with excitation and emission radiation.

FIG. 3 illustrates schematically the reflectance of a typical quantum dot structure without an antireflective shell area.

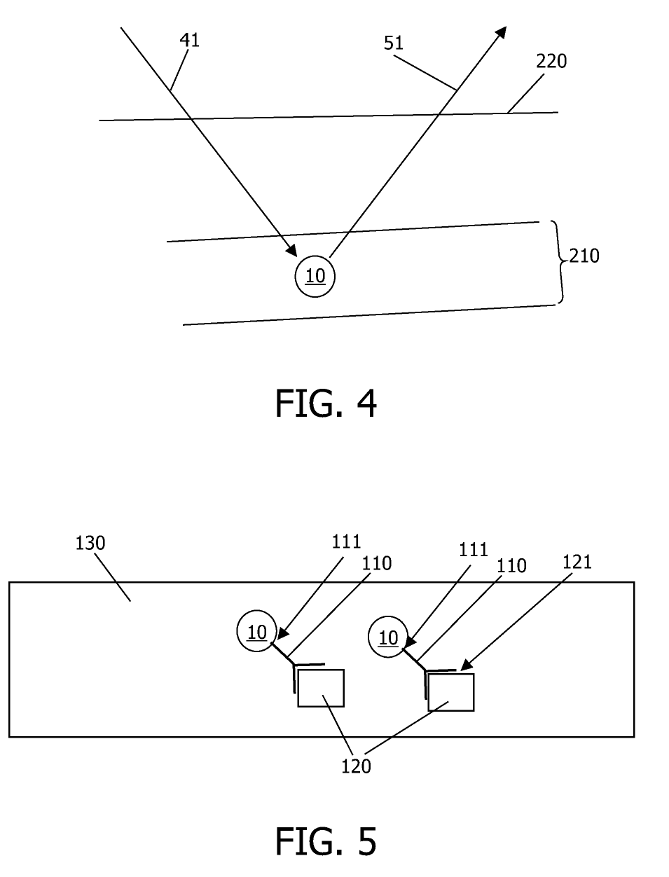

FIG. 4 illustrates schematically an example of an application of the luminescent particle as a contrast agent.

FIG. 5 illustrates schematically an example of an application of the luminescent particle in a biological assay.

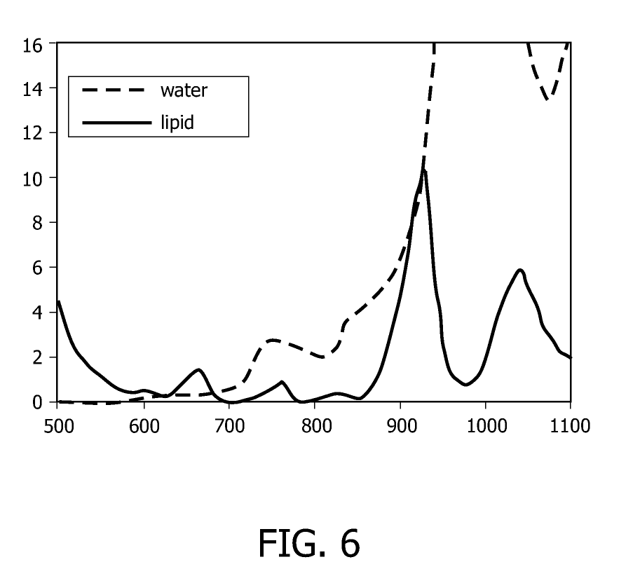

FIG. 6 illustrates schematically parts of the infrared absorption spectrum of main components of human tissue.

The present invention will be described with reference to particular embodiments and drawings, but the invention is not limited thereto. The drawings described are only schematic and are non-limiting. In the drawings, the dimensions of some of the elements may be exaggerated and not true to scale for reasons of clarity.

Where an indefinite or definite article is used in conjunction with a singular noun, e.g. “a”, “an”, “the”, this includes a plural of that noun unless specifically stated otherwise.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in sequences other than those described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in orientations other than those described or illustrated herein.

It is to be noted that the term “comprising” used in the present description and claims should not be interpreted as being restricted to the means listed thereafter; it does not exclude other elements or steps. Thus, the scope of the expression “a device comprising means A and B” should not be limited to devices consisting only of components A and B. It means that with respect to the present invention the only relevant components of the device are A and B.

FIG. 1 presents a cross section of a luminescent particle according to the present invention. The particle 10 comprises a core area 20 and a shell area 30. The core area 20 comprises a nanocrystal material 21 and the shell area 30 comprises a dielectric material 31. The shell area 30 preferably has a thickness D around the core area 20. The thickness D is preferably approximately constant around the core area 20.

In FIG. 2, the luminescent particle 10 according to the present invention is shown with an excitation radiation 41 and an emission radiation 51. Preferably, the luminescent particle 10 is used with infrared radiation for both excitation radiation 41 and emission radiation 51. The shell area 30 according to the present invention of the inventive luminescent particle 10 can especially have a fluorescent behavior.

FIG. 6 shows an example of the absorption characteristics of main components of human tissue for a portion of the electromagnetic spectrum. FIG. 6 shows the infrared portion of the electromagnetic spectrum. It can be seen, that there is an overall absorption minimum between approximately 600 and 900 nm wavelength. It is especially preferred to use the absorption window between 700 nm and about 800 nm wavelength for both the excitation radiation 41 and the emission radiation 51.

The core area 20 of the luminescent particle 10 is preferably realized with a nanocrystal material 21 comprising InP (indium phosphide). According to a preferred embodiment of the present invention, a structure of the nanocrystal material 21 is chosen such that is has a size suitable for an emission radiation 51 around an emission wavelength 50 of about 780 nm, where the infrared spectrum has minimum absorption for main components of human tissue, i.e. water and/or lipids. In this preferred embodiment of the present invention or in another embodiment of the present invention, the excitation of the luminescence behavior in the core area 20 can advantageously be produced at an excitation wavelength 40 of about 720 nm, where the other absorption minimum of the absorption window of FIG. 6 is located. According to the present invention, the use of indium phosphide as a nanocrystal material 21 for the core area 20 of the luminescent particle 10 is very much preferred because it is excellently suited for human application because of the possibility of optimally matching the absorption minima in the infrared spectrum for main components of human tissue (for the excitation wavelength 40 and/or for the emission wavelength 50). Furthermore, such a choice for the core area 20 makes it possible to use non-toxic materials for the nanocrystal material 21 of the core area 20 of the luminescent particle 10. Especially, it is possible to use a non-Cd-based nanocrystal material 21. This has the advantage that such a luminescent particle 10 will be more easily approved by national drug authorities for human applications, e.g. as a contrast agent or part of a contrast agent.

The shell area 30 of the luminescent particle 10 comprises a dielectric material 31 that provides the luminescent particle 10 with an antireflective coating 31 of the core area 20. This means that the optical properties of the luminescent particle 10 according to the present invention can be further improved by means of a layer of, for example, a dielectric material 31 such as TiO₂, GaP, or other ternary compounds such as InGaP₂. This decreases reflection losses considerably and increases the emission probability by up to a factor of four compared with the case where no antireflective coating 31 is provided around the core area 20. The dielectric material 31 of the shell area 30 has an electronic bandgap energy which is higher than the electronic bandgap energy of the nanocrystal material 21 of the core area 20. This makes the luminescent behavior of the core area 20 more effective.

The antireflective coating 31 or shell area 30 helps to avoid reflection and promotes emission. In a first approximation, the reflectance R at an interface between materials of different indices of refraction is given by the following formula:

R=(n−n′/n+n′)²

In this formula, n and n′ designate the respective indices of refraction of the two materials on either side of the interface.

Given the relative index of refraction of InP of around 3.3, one can calculate that the typical reflection in air is around 30%. Considering that this effect occurs twice, e.g. upon the excitation radiation 41 entering the luminescent particle 10 (from the outside of the luminescent particle 10 to the inside of the core area 20) and upon the emission radiation 51 exiting from the interior of the core area 20 towards the exterior of the luminescent particle 10, the losses due to reflection are given, for example, by the following formula:

(100%−R)*(100%−R)=(100%−30%)*(100%−30%)≈50%

Therefore, a properly chosen antireflective coating 31 of the core area 20 of the luminescent particle 10 will greatly improve the luminescence and/or luminescence properties of the nanocrystal material 21 of the core area 20.

FIG. 3 gives an example of the reflectance R for an example of an InP nanocrystal 21 for a different wavelength. The photon energy (unit: electron volts) is plotted on the abscissa, and the reflectance is plotted in relative units on the ordinate R.

In practice, the antireflective coating thickness is given by the following relationship:

$$D = \frac{λ}{4 {n}_{2}} = \frac{λ}{4 \sqrt{{n}_{1} {n}_{3}}}$$

$$with$$

$${n}_{2} = \sqrt{{n}_{1} {n}_{3}}$$

Here, λ is the wavelength and n₁n₃ is the product of a first index of refraction n₁ in the nanocrystal material 21 of the core area 20 and a third index of refraction n₃ at the exterior of the luminescent particle 10. For example, the use of the luminescent particle 10 inside the human body implies that the exterior of the luminescent particle 10 is, for example, blood, which has (by approximation) a third index of refraction n₃ of water which is about n₃=1.33. This implies that, with an observed wavelength of 750 nm (which is the average between an example of the excitation wavelength 40 (for example 720 nm) and an example of the emission wavelength 50 (for example 780 nm)), a wavelength of around 600 nm in blood is to be considered. A quarter of this is 150 nm. For a high-dielectric material 31 (i.e. with a comparably high second index of refraction n₂) of, for example, n₂≈30, this implies a coating thickness D of around 31 nm. By using a dielectric material 31 such as gallium phosphide (having a relative index of refraction of n₂=3.3) or a dielectric material 31 such as TiO₂ (with a relative index of refraction 2 of 2.5) the thickness D of the shell area 30 will be greater, for example around 95 or 110 nm, respectively. Yet, these particles are still sufficiently small for use as contrast agents in medical applications. Of course, materials with higher indices of refraction or higher dielectric constants yield smaller coating thicknesses D. As the thickness D of the dielectric material 31 of the shell area 30 of the luminescent particle 10 can only be (ideally) adapted to reduce the reflectance to the maximum for only one wavelength (excitation wavelength 40 or emission wavelength 50), it is preferred according to the present invention to adapt the thickness D to an average wavelength situated approximately centrally between the excitation wavelength 40 and the emission wavelength 50. Optimally, the dielectric material 31 of the shell area 30 is chosen such that its second index of refraction n₂ is approximately given as the square root of the product of the first and third indices of refraction n₁, n₃. As the shell area 30 also has other functions to fulfill (e.g. mechanical resistance, providing a binding place for other molecules to be connected to the luminescent particle, etc), it is clear that the optimum reduction of the reflectance R (i.e. the optimum degree of transmittance through the shell area 30) cannot always be achieved completely. Nevertheless, according to the invention, the transmittance of excitation radiation 41 and/or the transmittance of emission radiation 51 between the inside of the core area 20 and the outside of the luminescent particle 10 should be higher than 50% below the theoretical maximum of transmittance at a given thickness D and given first, second, and third indices of refraction n₁, n₂, n₃. Preferably, the transmittance of excitation radiation 41 and/or the transmittance of emission radiation 51 between the inside of the core area 20 and the outside of the luminescent particle 10 should be higher than 25% below the theoretical maximum of transmittance at a given thickness D and given first, second, and third indices of refraction n₁, n₂, n₃. Most preferably, the transmittance of excitation radiation 41 and/or the transmittance of emission radiation 51 between the inside of the core area 20 and the outside of the luminescent particle 10 should be higher than 10% below the theoretical maximum of transmittance at a given thickness D and given first, second, and third indices of refraction n₁, n₂, n₃.

FIG. 4 schematically shows an example of the use of the luminescent particle 10 as a contrast agent. A blood vessel 210 is provided under the outer surface of a patient's skin portion 220. The luminescent particle 10 is located inside the blood vessel 210 and irradiated by the excitation radiation 41, preferably from the exterior of the skin portion 220. The emission radiation 51 generated by the luminescent particle 10 is detected by a radiation detection means (not shown), also located preferably at the outside of the skin portion 220.

FIG. 5 schematically shows an example of the use of the luminescent particle 10 in a biological assay. A biological entity 120 is located at a fixed structure 130, e.g. a membrane or the like. The luminescent particle 10 is bound by a physical and/or chemical and/or biological binding 111 to a labeling substrate 110 specific to the biological entity 120 to be detected. The luminescent particle 10 and the labeling substrate 110 together form a complex. During the biological assay, the luminescent particle 10 (bound to the labeling substrate 110) (the complex) is exposed to the biological entity 120. Via a specific binding 121 between the biological entity 120 and the labeling substrate 110, the luminescent particle 10 is fixed to the biological entity 120 (and thus to the structure 130) and can be detected from the excitation and emission radiation (not shown in FIG. 5) by a radiation detection means (not shown).

Other methods or assays for detecting a biological entity 120 are obviously conceivable, e.g. without the use of a fixed structure 130 such as a membrane.

A biological entity 120 in the context of the present invention may be any of the following entities: one or a plurality of proteins, one or a plurality of nucleic acids, one or a plurality of fragments of a cell or different cells, or any other biological material.

1. Luminescent particle comprising a core area and a shell area, the core area being covered by the shell area, the core area conferring a luminescent behavior on the luminescent particle for at least one excitation wavelength and for at least one emission wavelength by means of a nanocrystal material, and the shell area being provided such that it realizes an antireflective coating of the core area.

2. Luminescent particle according to claim 1, wherein the core area is provided as a quantum dot structure realized by means of a non-Cd-based nanocrystal material.

3. Luminescent particle according to claim 1, wherein the core area is provided as a quantum dot structure realized by means of a non-toxic nanocrystal material.

4. Luminescent particle according to claim 1, wherein the shell area is provided as a non-Cd-based material and/or a non-toxic material.

5. Luminescent particle according to claim 1, wherein the core area is provided as a quantum dot structure realized by means of an InP-based-material.

6. Luminescent particle according to claim 1, wherein the excitation wavelength and the emission wavelength are provided in the near infrared portion of the electromagnetic spectrum.

7. Luminescent particle according to claim 6, wherein the excitation wavelength and the emission wavelength are provided in a spectral window of minimal infra red absorption in main components of human tissue, especially in human tissue liquid and/or lipid.

8. Luminescent particle according to claim 7, wherein the excitation wavelength and the emission wavelength are provided between 700 nm and 800 nm.

9. Luminescent particle according to claim 6, wherein the excitation wavelength is provided around 720 nm and/or the emission wavelength is provided around 780 nm.

10. Luminescent particle according to claim 6, wherein the excitation wavelength and the emission wavelength are separated by approximately 60 nm.

11. Luminescent particle according to claim 1, wherein the thickness (D) of the shell area is provided substantially homogeneously around the core area.

12. Luminescent particle according to claim 11, wherein the thickness (D) of the shell area is provided such that the reflectance of excitation radiation and/or the reflectance of emission radiation between the inside of the core area and the outside of the luminescent particle is comparatively low.

13. Luminescent particle according to claim 1, wherein the thickness (D) of the shell area is at least partially provided such that the reflectance of excitation radiation and/or the reflectance of emission radiation between the inside of the core area and the outside of the luminescent particle is comparatively low.

14. Luminescent particle according to claim 12, wherein the thickness (D) of the shell area is provided such that the transmittance of excitation radiation and/or the transmittance of emission radiation between the inside of the core area and the outside of the luminescent particle is higher than 50% below, preferably higher than 25% below, most preferably higher than 10% below the maximum transmittance for a given first index of refraction (n₁) of the nanocrystal material, a given second index of refraction (n₂) of the antireflective coating, and a given third index of refraction (n₃) of the environment of the luminescent particle.

15. Luminescent particle according to claim 1, wherein the shell area comprises a dielectric material.

16. Luminescent particle according to claim 15, wherein the dielectric material is provided as TiO₂ and/or GaP and/or InGaP₂ and/or other ternary compound(s).

17. Complex comprising a luminescent particle according to claim 1 and further comprising a labeling substrate.

18. Contrast agent comprising a luminescent particle according to claim 1.

19. Method of detecting a biological entity using a luminescent particle comprising a core area and a shell area, the core area being covered by the shell area, the core area conferring a luminescent behavior on the luminescent particle for at least one excitation wavelength and for at least one emission wavelength by means of a nanocrystal material, and the shell area being provided such that it realizes an antireflective coating of the core area, the method comprising the steps of:
forming a complex between the luminescent particle and a labeling substrate by means of a physical and/or a chemical and/or a biological binding,
using the labeling substrate for achieving a specific binding to the biological entity,
irradiating at least the complex of the luminescent particle and the labeling substrate with radiation of the excitation wavelength, and
detecting the biological entity by means of radiation emitted by the luminescent particle.

20. Method according to claim 19, wherein the complex of the luminescent particle and a labeling substrate is used in vivo.

21. Method according to claim 19, wherein the complex of the luminescent particle and the labeling substrate is used in vitro.

22. Use of the luminescent particle according to claim 1 in a biomedical assay and/or an in vitro application.

23. Use of a luminescent particle comprising a core area and a shell area, the core area being covered by the shell area, the core area conferring a luminescent behavior on the luminescent particle for at least one excitation wavelength and for at least one emission wave-length by means of a nanocrystal material, and the shell area being provided such that it realizes an antireflective-coating of the core area for producing a complex according to claim 17.
